(19) United States
(12) Patent Application Publication
(10) Pub. No.: US 2020/0188347 A1
(43) Pub. Date: Jun. 18, 2020

(54) ADMINISTRATION METHOD FOR FORMULATED CANNABINOID COMPOSITIONS

(71) Applicant: MD Biologics, LLC, Beverly Hills, CA (US)

(72) Inventors: Kia Michel, Beverly Hills, CA (US); Steve Ottersberg, Beverly Hills, CA (US); Mica Gross, Beverly Hills, CA (US); Babak Larian, Beverly Hills, CA (US); Kamran Jamshidinia, Beverly Hills, CA (US); Siamak Tabib, Beverly Hills, CA (US)

(73) Assignee: MD Biologics, LLC, Beverly Hills, CA (US)

(21) Appl. No.: 16/705,131

(22) Filed: Dec. 5, 2019

Related U.S. Application Data
(60) Provisional application No. 62/775,704, filed on Dec. 5, 2018.
(60) Provisional application No. 62/775,716, filed on Dec. 5, 2018.
(60) Provisional application No. 62/775,723, filed on Dec. 5, 2018.
(60) Provisional application No. 62/775,727, filed on Dec. 5, 2018.
(60) Provisional application No. 62/775,732, filed on Dec. 5, 2018.

(51) Int. Cl. A61K 31/352; A61K 31/05; A61K 31/12; A61K 36/324; A61K 36/67; A61K 31/685; A61K 9/00; A61K 36/185

(52) U.S. Cl. CPC A61K 31/352; A61K 31/05; A61K 31/12; A61K 36/185; A61K 36/67; A61K 31/685; A61K 9/0053; A61K 36/324

(57) ABSTRACT

A method of oral administration of cannabinoids includes the steps of administering at least one first softgel containing a non-psychoactive cannabinoid and no psychoactive cannabinoids for a first administration cycle to the subject, administering at least one second softgel containing a first cannabinoid mix having the non-psychoactive cannabinoid and a psychoactive cannabinoid for a second administration cycle to the subject, wherein the first cannabinoid mix has no more than 10% on a w:w basis of the psychoactive cannabinoid, administering at least one third softgel containing a second cannabinoid mix having the first non-psychoactive cannabinoid and the psychoactive cannabinoid for a third administration cycle to the subject, wherein the second cannabinoid mix has no more than 50% on a w:w basis of the psychoactive cannabinoid.

### FIELD OF THE INVENTION

The present invention pertains to methods of oral administration of cannabinoids, and particularly methods of administration of particular cannabinoid formulations.

### BACKGROUND OF THE INVENTION

Oral administration of cannabinoids normally results in less than 3% of bioactive cannabinoids reaching the blood stream of a patient or subject. This reduction is due primarily to first pass metabolism. During first pass metabolism hepatic enzymes, including cytochrome p450 enzymes, degrade the cannabinoids that pass out from the digestive tract. These enzymes are heme containing proteins that function as monooxygenases.

Individuals may react differently to cannabinoids. The reasons for this are yet to be fully understood. It is known that some people lack the ability to produce cannabinoid oxidizing enzymes in the liver in sufficient amount to efficiently degrade the cannabinoids. Others produce excessive amounts of the oxidative enzymes. Additionally, various pharmaceuticals can interact with the liver and affect the production and efficacy of oxidative enzymes. Diet, gut microbiome, genetic expressions, hepatic toxification, hepatic disease and numerous other factors can affect the production and efficacy of hepatic enzymes in degrading cannabinoids that are orally consumed. A potentially a large variance across a population of subjects and patients can be seen in the bio-effects of cannabinoids.

This large variance of individual reactions to orally delivered cannabinoids has caused health practitioners, and physicians in particular, to consider how to develop a common cannabinoid administration protocol to serve a variety of people in a safe and effective manner.

### SUMMARY OF THE INVENTION

The present invention includes a method of oral administration of cannabinoids to a subject to enable the subject to become accustom to the usage and effects of psychoactive cannabinoids. The method includes administering at least one first softgel containing a non-psychoactive cannabinoid and no psychoactive cannabinoids for a first administration cycle to the subject. Next the method includes administering at least one second softgel containing a first cannabinoid mix having the non-psychoactive cannabinoid and a psychoactive cannabinoid for a second administration cycle to the subject, wherein the first cannabinoid mix has no more than 10% on a w:w basis of the psychoactive cannabinoid. Next the method includes administering at least one third softgel containing a second cannabinoid mix having the first non-psychoactive cannabinoid and the psychoactive cannabinoid for a third administration cycle to the subject, wherein the second cannabinoid mix has no more than 50% on a w:w basis of the psychoactive cannabinoid.

The non-psychoactive cannabinoid is preferably cannabidiol and the psychoactive cannabinoid is tetrahydrocannabinol. In alternate embodiments, the non-psychoactive cannabinoid includes CBG (Cannabigerol), CBD (Cannabidiol), CBC (Cannabichromene), CBGV (Cannabigerivarin), THCV (Tetrahydrocannabivarin), CBDV (Cannabidivarin), CBCV (Cannabichromevarin) or combinations thereof.

The method of the present invention further includes administering at least one fourth softgel for a fourth administration cycle containing one psychoactive cannabinoid and no non-psychoactive cannabinoid. In an embodiment targeting inflammation and pain management, each of the first softgel, second softgel, third softgel and fourth softgel includes curcumin, boswellia, Black Pepper extract, phosphatidyl choline, and myrcene.

In an alternate embodiment, the softgel encloses an ingredient selected from the group consisting of: mycrene, phosphatidyl choline, black pepper extract, boswellia, curcumin, and combinations thereof.

The softgel is a gelatin enclosure, similar to a capsule, having a liquid center. The active ingredients are in the liquid center.

In one embodiment, the first administration cycle includes delivering three 500 mg softgels daily for at least one week, the second administration cycle includes delivering three 500 mg softgels daily for at least one week and the cannabinoid mix delivered during the second administration cycle includes 90% cannabidiol and 10% tetrahydrocannabinol. The third administration cycle includes delivering three 500 mg softgels daily for at least one week and the cannabinoid mix delivered during the second administration cycle includes 50% cannabidiol and 50% tetrahydrocannabinol.

In one embodiment of the invention, all of the softgels used in the administration cycles share 167 mg of curcumin+/−20%, 3.3 mg of black pepper extract+/−20% and 83 mg of boswellia+/−20%.

In one embodiment the softgels are administered to a subject such as a human or mammal according to an escalated administration method.

### First Formulation

An example administration method includes providing a first formulation including a first cannabinoid, the first cannabinoid being non-psychoactive. In one embodiment the first cannabinoid is Cannabidiol (CBD). Initiating an administration method with a non-psychoactive cannabinoid reduces risks to a subject that may have an adverse mental, physical or emotional reaction to psychoactive cannabinoids. Initiating an administration method with a non-psychoactive cannabinoid also enables a subject to better accommodate cannabinoids. A further benefit is to gauge the intensity and duration of the cannabinoid administered so that the administration protocol may be adapted in response to the intensity and duration of the administered cannabinoid.

Preferably, the administration of first formulation is administered to a subject via oral delivery for a predetermined period. For example, a two week period, is ideal. Administration can be self-administration, or with assistance from a health practitioner.

During this first two week period only non-psychoactive cannabinoids are administered. More preferably, only one non-psychoactive cannabinoid is administered. In one embodiment, the non-psychoactive cannabinoid is Cannabidiol (CBD). Trace psychoactive cannabinoids may be present but having no bio effect when derived from full spectrum extract of Cannabis sativa 1.

During this first two week period the cannabinoid formulation is administered three times daily, increasing from 10 mg CBD three times daily to 20 mg CBD, or more, three times daily. It can be appreciated that the formulation may contain any of a variety of non-psychoactive cannabinoids.

Cannabinoid is defined as any compound or substance that directly influences either the CB1, CB2, or other cannabinoid receptors including receptors yet to be discovered in humans.

Cannabinoids include phytocannabinoids, endocannabinoids or synthetic cannabinoids. Synthetic cannabinoids can be made from yeast, or other microbes, or synthesized chemically. Particular cannabinoids specified herein include both the acid and non-acid forms of the molecule unless otherwise specified.

### Second Formulation

The method includes administering a second formulation including a mixture of cannabinoids, and that mixture includes a psychoactive cannabinoid.

The period for administration of the mixture of cannabinoids is at least a four week period according to an escalated administration schedule. This mixture of cannabinoids includes tetrahydrocannabinol (THC).

This second formulation is administered after the first formulation administration cycle is completed. Preferably, the second formulation includes more CBD than THC and the concentration of these cannabinoids varies over time to steadily increase the THC content of the cannabinoid mix. In this way the subject becomes accustom to psychoactive THC, and should mental, physical, or emotional adverse effects can be monitored and reported to a health care professional. Mental adverse effects include hyperactive thought patterns or loss of concentration abilities. Emotional adverse effects may include fear or hyper anxiety. Physical adverse effects may include hyperactivity or lethargy.

More preferably, the second formulation includes less than 10% THC in the cannabinoid mix, for example 10 mg CBD and 1 mg THC. This second formulation is administered three times a day for 1-2 weeks.

### Third Formulation

After the cycle of administration for the second formulation is complete, the method escalates the concentration of THC to between 10-20% of the cannabinoid mix. For example, 8 mg CBD and 2 mg of THC is delivered three times a day for 1 week.

### Forth Formulation

After the cycle of administration of the third formulation is complete, the method further includes escalating the concentration of THC to between 20-50% of the cannabinoid mix. For example, the fourth formulation includes 5 mg CBD and 5 mg THC administered three times a day for 2 additional weeks.

### Fifth Formulation

After the cycle of administration of the third formulation is complete, the method further includes escalating the concentration of THC to between 50-100% of the cannabinoid mix. For example, the fourth formulation includes 10 mg THC administered three times a day for 2 additional weeks, or as long as necessary. The frequency of delivery can increase more than times per day. In a variation of this embodiment, the fifth formulation may not include any CBD.

The subject, upon recognizing efficacy, has the option to continue an administration schedule that is deemed effective, without further escalation.

The dosage escalation method enables a subject to become accustomed to cannabinoids so that the subject may function normally during the treatment process, and if any adverse reactions, or adverse drug interactions with other drugs occur, such will be less impactful and more readily managed. Another benefit of the administration schedule is that a subject may find that the desired effect such as pain management may be achieved with CBD only, or a moderate to low dose of THC and CBD in combination.

In one embodiment, the oral delivery is ideally accomplished with a softgel. It can be appreciated that the present administration method can be applied with a tablet, capsule, tincture, liquid spray, tablet, or other appropriate oral delivery method.

### DETAILED DESCRIPTION

### Example Formulations:

The formulations of the present invention include numerous bioactive ingredients other than cannabinoids, and the method of the invention may include varying the administration of these ingredients to achieve the purposes of the invention.

In one embodiment of the invention, the formulations of the present invention are achieved with a whole plant extract, so numerous additional cannabinoids and terpenes can be present in both trace and bioactive amounts. In an alternate embodiment, the cannabinoids used are isolated cannabinoids and precise amounts of isolated terpenes, or other ingredients, are added. The following examples are tabulated and it can be appreciated that the ingredients on a percentage basis can vary by +/−20%.

TABLE 1

| | TOTAL DOSE | Per Softgel |
|---|---|---|
| SOFT GELS | (mg) | (mg) |
| THC | 2.5 | 0.83 |
| CBD | 30 | 10.00 |
| Curcumin | 500 | 166.67 |
| Boswellia | 250 | 83.33 |
| Black Pepper extract | 10 | 3.33 |
| Phosphatidyl choline | 200 | 66.67 |
| Myrcene | 1 | 0.33 |
| MCT | 480 | 160.00 |
| Total Fill | 1473.5 | 491.17 |

TABLE 2

| | TOTAL DOSE | Per Softgel |
|---|---|---|
| SOFT GELS | (mg) | (mg) |
| CBD | 30 | 15.00 |
| 5-HTP | 100 | 50.00 |
| Magnesium Glycinate | 100 | 50.00 |
| Passionflower extract | 50 | 25.00 |
| Chamomile Extract | 200 | 100.00 |
| Myrcene | 1 | 0.50 |
| MCT | 500 | 250.00 |
| TOTAL FILL | 981 | 490.50 |

TABLE 3

| | TOTAL DOSE | Per Softgel |
|---|---|---|
| SOFT GELS | (mg) | (mg) |
| CBD | 30 | 10.00 |
| Curcumin | 500 | 166.67 |
| Boswellia | 250 | 83.33 |
| Black Pepper extract | 10 | 3.33 |
| Phosphatidyl choline | 200 | 66.67 |
| Myrcene | 1 | 0.33 |
| MCT | 480 | 160.00 |
| Total Fill | 1471 | 490.33 |

TABLE 4

| | TOTAL DOSE | Per Softgel |
|---|---|---|
| SOFT GELS | (mg) | (mg) |
| THC | 2.5 | 1.25 |
| CBD | 30 | 15.00 |
| 5-HTP | 100 | 50.00 |
| Magnesium Glycinate | 100 | 50.00 |
| Passionflower extract | 50 | 25.00 |
| Chamomile Extract | 200 | 100.00 |
| Myrcene | 1 | 0.50 |
| MCT | 500 | 250.00 |
| TOTAL FILL | 983.5 | 491.75 |

TABLE 5

| | TOTAL DOSE | Per Softgel |
|---|---|---|
| SOFT GELS | (mg) | (mg) |
| THC | 2.5 | 0.83 |
| CBD | 30 | 10.00 |
| Curcumin | 500 | 166.67 |
| Boswellia | 250 | 83.33 |
| Black Pepper extract | 10 | 3.33 |
| Phosphatidyl choline | 200 | 66.67 |
| Myrcene | 1 | 0.33 |
| MCT | 480 | 160.00 |
| Total Fill | 1473.5 | 491.17 |

The tables 1-5 above show various examples of formulations in accordance with the present invention. Each of the various ingredients can vary by +/−20%, and they are administered within a single 500 mg softgel. A single dose may include one or more softgels. The above table anticipates a dose including three softgels and sets forth the total dose assuming that three softgels constitute a dose, and the tables lists the ingredients of each single softgel.

Table 4 lists ingredients that are particularly useful for improving sleep. These ingredients include magnesium glycinate, passionflower extract and chamomile extract in each softgel administered in accordance with the method of the present invention. These ingredients, in combination, have been found to vastly improve sleep patterns in willing subjects.

The cannabinoid concentration can vary. For example, a 500 mg softgel can be developed to have between 1-50 mg of cannabinoid. For example, 1, 2, 5 or 10 mg THC, or CBD, or both, and can be presented in a single softgel. The softgel can also include any cannabinoid with the array of synergistic ingredients indicated in table 1.

Curcumin is a diaryheptanoid belonging to the group of curcuminoids. Curcumin is a tautomeric compound that exists in enolic form in organic solvents. It is both unstable and minimally bioavailable in its pure form. However, when combined in the present invention in the softgel the curcumin is bioavailable and shelf stable. The curcumin cooperates with the cannabinoids to achieve anti-inflammatory effects in vivo. Curcumin is known to interact with cytochrome p-450 heptatic enzymes. This interaction may reduce the effects of first pass metabolism of the cannabinoids in the liver and bolster the efficacy of cannabinoids.

The phosphatidylcholine utilized in the present formulation as a lipid-solvent that provides a mechanism to enable curcumin and cannabinoids to achieve a miscible lipid solution in the manufacture of the softgel. The phosphatidylcholine further enhances bioavailability of the cannabinoids and curcumin.

The black pepper extract provides is high in various synergistic terpenes including f3-caryophyllene, a sesquiterpene, and f3-myrcene, a monoterpene. The boswellia provides an additional terpene mix.

While many terpenes are found naturally in Cannabis sativa, and in a whole plant extracts of Cannabis sativa, they can be isolated from a variety of natural and synthetic sources, used to bolster desired effects of cannabinoids, and minimize less desirable effects such as reducing anxiety experienced by some subjects. Isolated myrcene can be added to the formulation to further bolster the desirable synergistic effects.

The formulations of the present invention include numerous bioactive ingredients other than cannabinoids, and the method of the invention may include varying the administration of these ingredients to achieve the purposes of the invention.

Cannabinoids include the following common cannabinoids, which are found in whole plant extracts in their acid forms. Cannabinoids also include acid form cannabinoids and analogues thereto. The following are the non-psychoactive cannabinoids as defined herein: CBG (Cannabigerol), CBD (Cannabidiol), CBC (Cannabichromene), CBGV (Cannabigerivarin), THCV (Tetrahydrocannabivarin), CBDV (Cannabidivarin), CBCV (Cannabichromevarin). In various aspects of the invention, these cannabinoids can be substituted for CBD to help a patient or subject become accustom to use of cannabinoids prior to, or during, administration of THC which is considered psychoactive.

Preferably the formulations of the present invention are achieved with a whole plant extract, so numerous additional cannabinoids and terpenes can be present in both trace and bioactive amounts. In an alternate embodiment, the cannabinoids used are isolated cannabinoids and precise amounts of isolated terpenes are added.

We claim:

1. A method of oral administration of cannabinoids to a subject, comprising:
administering at least one first softgel containing a non-psychoactive cannabinoid and no psychoactive cannabinoids for a first administration cycle to the subject;
administering at least one second softgel containing a first cannabinoid mix having the non-psychoactive cannabinoid and a psychoactive cannabinoid for a second administration cycle to the subject, wherein the first cannabinoid mix has no more than 10% on a w:w basis of the psychoactive cannabinoid;
administering at least one third softgel containing a second cannabinoid mix having the first non-psychoactive cannabinoid and the psychoactive cannabinoid for a third administration cycle to the subject, wherein the second cannabinoid mix has no more than 50% on a w:w basis of the psychoactive cannabinoid; and
wherein the non-psychoactive cannabinoid is cannabidiol and the psychoactive cannabinoid is tetrahydrocannabinol.

2. The method of oral administration of cannabinoids as set forth in claim 1 further comprising administering at least one fourth softgel for a fourth administration cycle containing one psychoactive cannabinoid and no non-psychoactive cannabinoid.

3. The method of oral administration of cannabinoids as set forth in claim 2, wherein the each of the first softgel, second softgel, third softgel and fourth softgel includes curcumin.

4. The method of oral administration of cannabinoids as set forth in claim 2, wherein the each of the first softgel, second softgel, third softgel and fourth softgel includes boswellia.

5. The method of oral administration of cannabinoids as set forth in claim 2, wherein the each of the first softgel, second softgel, third softgel and fourth softgel includes Black Pepper extract.

6. The method of oral administration of cannabinoids as set forth in claim 2, wherein the each of the first softgel, second softgel, third softgel and fourth softgel includes phosphatidyl choline.

7. The method of oral administration of cannabinoids as set forth in claim 2, wherein the each of the first softgel, second softgel, third softgel and fourth softgel includes myrcene.

8. The method of oral administration of cannabinoids as set forth in claim 1, wherein at least one of said first softgel, second softgel, third softgel and fourth softgel includes an ingredient selected from the group consisting of: mycrene, phosphatidyl choline, black pepper extract, boswellia, curcumin, and combinations thereof.

9. The method of oral administration of cannabinoids as set forth in claim 1, wherein the first administration cycle includes delivering three 500 mg softgels daily for at least one week.

10. The method of oral administration of cannabinoids as set forth in claim 9 wherein the second administration cycle includes delivering three 500 mg softgels daily for at least one week and the cannabinoid mix delivered during the second administration cycle includes 90% cannabidiol and 10% tetrahydrocannabinol.

11. The method of oral administration of cannabinoids as set forth in claim 10 wherein the third administration cycle includes delivering three 500 mg softgels daily for at least one week and the cannabinoid mix delivered during the second administration cycle includes 50% cannabidiol and 50% tetrahydrocannabinol.

12. A method of oral administration of cannabinoids to a subject, comprising:
administering at least one first softgel containing a non-psychoactive cannabinoid and no psychoactive cannabinoids for a first administration cycle to the subject;
administering at least one second softgel containing a first cannabinoid mix having the non-psychoactive cannabinoid and a psychoactive cannabinoid for a second administration cycle to the subject, wherein the first cannabinoid mix has no more than 10% on a w:w basis of the psychoactive cannabinoid;
administering at least one third softgel containing a second cannabinoid mix having the first non-psychoactive cannabinoid and the psychoactive cannabinoid for a third administration cycle to the subject, wherein the second cannabinoid mix has no more than 50% on a w:w basis of the psychoactive cannabinoid; and
wherein the psychoactive cannabinoid is tetrahydrocannabinol and the non-psychoactive cannabinoid is derived from a full spectrum extract of Cannabis sativa 1.

13. The method of oral administration of cannabinoids as set forth in claim 12 further comprising administering at least one fourth softgel for a fourth administration cycle containing one psychoactive cannabinoid and no non-psychoactive cannabinoid.

14. The method of oral administration of cannabinoids as set forth in claim 12, wherein at least one of said first softgel, second softgel, third softgel and fourth softgel includes an ingredient selected from the group consisting of: mycrene, phosphatidyl choline, black pepper extract, boswellia, curcumin, and combinations thereof.

15. The method of oral administration of cannabinoids as set forth in claim 12, wherein the first administration cycle includes delivering three 500 mg softgels daily for at least one week.

16. The method of oral administration of cannabinoids as set forth in claim 15 wherein the second administration cycle includes delivering three 500 mg softgels daily for at least one week and the cannabinoid mix delivered during the second administration cycle includes 90% cannabidiol and 10% tetrahydrocannabinol.

17. The method of oral administration of cannabinoids as set forth in claim 15 wherein the third administration cycle includes delivering three 500 mg softgels daily for at least one week and the cannabinoid mix delivered during the second administration cycle includes approximately 50% cannabidiol and 50% tetrahydrocannabinol.

18. The method of oral administration of cannabinoids as set forth in claim 12, wherein the each of the first softgel, second softgel, third softgel and fourth softgel includes 167 mg of curcumin+/−20%.

19. The method of oral administration of cannabinoids as set forth in claim 12, wherein the each of the first softgel, second softgel, third softgel and fourth softgel includes between 3.3 mg of black pepper extract+/−20%.

20. The method of oral administration of cannabinoids as set forth in claim 12, wherein the each of the first softgel, second softgel, third softgel and fourth softgel includes between 83 mg of boswellia+/−20%.
